(19) United States
(12) Patent Application Publication
(10) Pub. No.: US 2014/0378796 A1
(43) Pub. Date: Dec. 25, 2014

(54) SYSTEM AND METHOD FOR NEEDLE NAVITATION USING PA EFFECT IN US IMAGING

(71) Applicant: KONINKLIJKE PHILIPS N.V., EINDHOVEN (NL)

(72) Inventors: Yinan Chen, Shanghai (CN); Jingping Xu, Shanghai (CN); Junbo Li, Shanghai (CN); Yunrong Zhang, Shanghai (CN)

(21) Appl. No.: 14/369,664

(22) PCT Filed: Dec. 27, 2012
(86) PCT No.: PCT/IB2012/057760

§ 371 Date: Jun. 27, 2014

(30) Foreign Application Priority Data
Dec. 30, 2011 (CN) PCT/CN2011/085050

(51) Int. Cl. A61B 19/00; A61B 8/08; A61B 17/34; A61B 5/1455; A61B 5/145; A61B 5/00; A61B 5/1459

(52) U.S. Cl. CPC A61B 19/5244; A61B 5/0095; A61B 8/0841; A61B 5/0035; A61B 5/1459; A61B 5/14552; A61B 5/14546; A61B 5/7278; A61B 5/743; A61B 17/3478; A61B 2019/5255; A61B 2019/5263
USPC 600/328; 600/424

(57) ABSTRACT

The present invention provides a monitoring system, which comprises a novel needle, and an optical signal generating device, wherein at least one optical signal output of the optical signal generating device is coupled to the optical core of the needle, and it further comprises an ultrasound (US) transducer,and a processor adapted to direct the US transducer to transmit an US signal into a region of a subject in which the needle is moving and receive an US signal reflected in the region in response to the transmitted US signal in a US measurement sub-cycle of a measurement cycle, and to direct the optical signal providing device to transmit an optical signal having a unique wavelength from the dome of the needle into an area of the region and direct the US transducer to receive a photo-acoustic (PA) signal induced in the area in response to the optical signal in each of at least one PA measurement sub-cycle of the measurement cycle, and reconstruct an US image from the US signal received in the US measurement sub-cycle.

### FIELD OF THE INVENTION

The invention relates to needle navigation, and particularly to a system and method for monitoring needle movement inside a subject by using the photo-acoustic (PA) effect in ultrasound (US) imaging.

### BACKGROUND OF THE INVENTION

A navigation system can assist the physician performing an intervention in guiding various phases of needle placement in a subject. One of the technical challenges for a navigation system is to maintain accuracy. The data used before and during the intervention should provide the physician with all necessary clues at sensible moments of the intervention and with sufficient accuracy, so that the physician knows where the needle is in the subject, and where the planned final placement of the needle will be. Hence, the needle location, the road mapping elements from the planning, the diagnostic elements (tumor location and shape, liver anatomy, including shape and vasculature), the US data of the live viewing, etc: all these elements should be localized with respect to each other rapidly and with sufficient accuracy.

Tracking devices are essential components of an image-guided interventional therapy system. Early tracking systems were mechanical digitizers, and then optical tracking systems were adopted due to their high accuracy and relatively large workspace. A line-of-sight between the tracking device and the instrument to be tracked is needed for optical tracking systems, and this limits the range of application of the optical tracking systems in real clinical situations. Electromagnetic (EM) tracking systems have been developed which do not require a line-of-sight. The sensor coil, actually the EM tracker entity, induces a varying voltage which is used by the measurement system to calculate the position and orientation of the object. These low strength magnetic fields can safely pass through human tissue, and measure the location of an object without the line-of-sight constraints. Therefore, the navigation needle is always equipped with a miniature EM tracker close to its tip to keep track of the location and orientation of the needle punching into the subject. One of the limitations for the use of EM trackers is the strict operating condition that no magnetically susceptible materials are placed in proximity to the magnetic field generated. This is often challenging due to the common occurrence of such materials in the hospital bed, needle, surgical tools, ultrasound probe and even the holder of the magnetic field.

One of the new and promising techniques for non-invasive and non-destructive imaging is a hybrid system known as PA imaging. It relies on the PA effect, which is a phenomenon whereby the absorbed energy from a very short laser light is transformed into kinetic energy of the sample by energy exchange processes, and then results in local heating and thus generates a pressure wave in the frequency range of ultrasound. The PA effect is the generation of acoustic waves by the absorption of electromagnetic energy.

In PA imaging, non-ionizing laser pulses are delivered into biological tissues, absorbed by tissue chromophores, and then converted into heat. This leads to transient thermo-elastic expansion, followed by the excitation of the spatial emissive distribution of the acoustic transient pressure inside the tissue, thus acting as the initial source of the acoustic waves. The generated acoustic waves propagate through the underlying tissue to the surface where an US transducer is placed to receive these PA wave signals. These wave signals are used to reconstruct the absorbed energy distribution, and finally to determine the distribution of optical absorption coefficients for the tissue. In summary, PA images indicate optical contrast in the US resolution for soft biological tissues.

The latest reports show that PA imaging has already been applied to the problem of image-guided metal needle tracking, and the validity has been evaluated using phantom tissue (Su, J., Karpiouk, A., Wang, B., and Emelianov, S., “Photoacoustic imaging of clinical metal needles in tissue,” J. Biomed. Opt. 15(2), 021309 (2010); Kim, C., Erpelding, T. N., Maslov, K., Jankovic, L., Akers, W. J., Song, L., Achilefu, S., Margenthaler, J. A., Pashley, M. D., and Wang, L. V., “Handheld array-based photoacoustic probe for guiding needle biopsy of sentinel lymph nodes,” J. Biomed. Opt. 15(4), 046010 (2010)). A high-contrast image of commonly used metal needles can be obtained by PA imaging combined with current US imaging methods. The published technology relies on the high absorption of the metal matter, so the metal needle relative to the background is clearly shown in the PA image. All these methods utilize multi-fiber optical bundles interfaced around the transducer outside the organism. The arrangement of the fiber bundles should enable both light and sound to be delivered along the same plane, which increases the complexity of the device.

### SUMMARY OF THE INVENTION

The present invention provides a system for monitoring the location of a needle inside a region of a subject and simultaneously measuring physiological properties in the tissue around the needle tip, such as oxyhemoglobin, deoxyhaemoglobin, carbonized tissue, oxygen saturation, as the needle moves to the potentially different biological tissues in the subject.

According to one aspect of the present invention, a novel needle is provided. By using the needle to generate PA signals in tissue area to be punctured, the proposed monitoring system may obtain the conventional US image, the location of the needle tip, and pathological information such as concentration of hemoglobin and oxygen saturation of blood in the area all at once. The needle comprises: a shell with needle shape; an optical dome disposed at a tip of the shell to form an inner needle space closed at the end of the tip; and an optical core built in the inner needle space, wherein the optical dome forms a lens for radiating optical signals transferred by the optical core out of the needle.

According to an embodiment of the present invention, the needle is composed of four layers which, from outside to inside, are the shell, the buffer, the cladding and the optical core, respectively, wherein the optical core comprises one or more fibers.

According to one aspect of the present invention, there is provided a monitoring system comprising

the above mentioned needle;

an optical signal generating device, wherein at least one optical signal output of the optical signal generating device is coupled to the optical core;

an ultrasound (US) transducer; and

a processor adapted to direct the US transducer to transmit a US signal into a region of a subject in which the needle is moving, and receive a US signal reflected in the region in response to the transmitted US signal in a US measurement sub-cycle of a measurement cycle, and direct the optical signal providing device to transmit an optical signal having a unique wavelength from the dome of the needle into an area of the region and direct the US transducer to receive a PA signal induced in the area in response to the optical signal in each of at least one photo-acoustic (PA) measurement sub-cycle of the measurement cycle, and reconstruct a US image from the US signal received in the US measurement sub-cycle.

Through defining the specific timing of the measurement cycle in which the US transducer and the needle operate, the conventional US imaging and the PA imaging related functions may be implemented together without interference to each other.

According to an embodiment of the present invention, the processor is further adapted to direct the optical signal generating device to transmit a first optical signal having a first wavelength from the dome of the needle into the area of the subject in one of the at least one PA measurement sub-cycle, direct the US transducer to receive a PA signal induced in the area in response to the first optical signal in the PA measurement sub-cycle, reconstruct a PA image from the received PA signal, and fuse the PA image and the US image to achieve a fused image to be displayed.

Through radiating a first optical signal having a first wavelength from the dome of the needle into the area in front of the needle tip, the location of the needle tip is presented in the reconstructed PA image and enhanced in the US image by fusing both images. By focusing on the location of the needle tip, the computation load may be reduced.

According to an embodiment of the present invention, the first wavelength is selected to be in a range of 200-400 nanometer (nm) in which the optical energy is attenuated fast in biological tissue and thus the needle tip location in the image is compressed into a spot, so that the location is more accurate.

According to an embodiment of the present invention, the processor is further adapted to direct the optical signal generating device to transmit a second optical signal having a second wavelength from the dome of the needle into the area of the subject in one of the at least one PA measurement sub-cycle, direct the US transducer to receive a PA signal induced in the area in response to the second optical signal in the PA measurement sub-cycle, determine the concentration of a chromophore, whose absorption property depends on the second wavelength, in the area according to the received PA signal, compare the concentration of the chromophore determined at the current needle tip location to at least one of those determined at previous needle tip locations, and trigger an alarm to a viewer when the comparison indicates a sudden change of the concentration of the chromophore determined at the current needle tip location.

The main effective response matter to the PA phenomena is the chromophores in tissue. It is known that all chromophores, such as water, oxy-hemoglobin, deoxy-hemoglobin, lipid, cytochrome oxidase and melanin in biological tissues have characteristic spectroscopic optical absorption features. These are essentially the fingerprints which allow these chromophores to be uniquely identified. Various tissues, containing different concentrations of chromophores, also show different optical absorption spectra. Through alarming a viewer that a potentially different tissue area may be touched, this embodiment is beneficial to obtain a non-destructive puncture.

According to an embodiment of the present invention, the second wavelength is selected in a range of 400-600 nm, the chromophore is hemoglobin, and the processor is further adapted to trigger an alarm when the comparison indicates a sudden increase of the concentration of the chromophore determined at the current needle tip location, and present the concentrations of the chromophore determined at different needle tip locations on the US image.

Optical absorption in biological tissues can be due to endogenous chromophores such as melanin or exogenously delivered contrast agents. Usually, blood has an order of magnitude larger absorption than surrounding tissues, so there is sufficient endogenous contrast in a PA image to visualize blood vessels as well as insight of tumor microenvironment, and hemodynamics, etc. This embodiment is beneficial to obtain a non-destructive puncture. PA imaging with the benefit of deep penetration and high resolution can enable clinicians to avoid contact with important blood vessels (hepatic artery, portal vein, etc) in advance of co-localization, ultimately providing a strong tool for navigation.

According to an embodiment of the present invention, the processor is further adapted to direct the optical signal generating device to transmit consecutively a third optical signal having a third wavelength and a fourth optical signal having a fourth wavelength from the dome of the needle into the area of the subject in two of the at least one PA measurement sub-cycle, direct the US transducer to consecutively receive PA signals induced in the area in response to the third and fourth optical signals in the two PA measurement sub-cycles, determine concentration of oxyhemoglobin (HbO2) and concentration of deoxyhemoglobin (Hb) in the area according to the received PA signals, determine oxygen saturation (SO2) of blood in the area according to the concentration of HbO2 and the concentration of Hb, and present the SO2 on the US image to be displayed.

According to an embodiment of the present invention, the molar extinction coefficients of Hb and HbO2 in the third and fourth wavelengths enable the concentrations of Hb and HbO2 to be precisely derived by the optical absorption values measured from PA signals.

According to an embodiment of the present invention, the third and fourth wavelengths are selected to be 940 nm and 660 nm, respectively.

Through utilizing multiple wavelengths, pathological information such as SO2 of blood may be provided together with the needle tip location and chromophore concentration on the fused image.

According to an aspect of the present invention, there is provided a method of monitoring during the time that a needle as mentioned above moves in a region of a subject, the method comprising:

applying an ultrasound (US) signal to the region in an US measurement sub-cycle of a measurement cycle, and receiving an US signal reflected in the region in response to the applied US signal;

in each of at least one photo-acoustic (PA) measurement sub-cycle of the measurement cycle, applying an optical signal having a different wavelength from the dome of the needle into an area of the subject and receiving a PA signal induced in the area in response to the optical signal; and
reconstructing an US image from the US signal received in the US measurement sub-cycle.

According to another aspect of the present invention, there is provided a computer program product, comprising machine executable instructions which, when executed on a machine, cause the machine to perform the above mentioned methods.

Other objects and advantages of the present invention will become more apparent and will be easily understood with reference to the description given in combination with the accompanying drawings.

### DESCRIPTION OF THE DRAWINGS

The present invention will be described and explained hereinafter in more detail in combination with embodiments and with reference to the drawings, in which:

FIG. 1 is a schematic diagram of a system for monitoring the location of a needle and physiological indices of tissue into which the needle is to be inserted, during needle movement in a subject in accordance with an embodiment of the present invention;

FIG. 2 is a schematic diagram of the structure of a needle employed in the monitoring system in accordance with an embodiment of the present invention;

FIG. 3 is a flowchart of the method of navigating the insertion of a needle in a subject in accordance with an embodiment of the present invention;

FIG. 4 is a diagram showing the different absorption spectra represented in terms of molar extinction coefficients.

The same reference signs in the figures indicate similar or corresponding features and/or functionalities.

### DETAILED DESCRIPTION

The embodiment of the present invention will be described hereinafter in more detail with reference to the drawings.

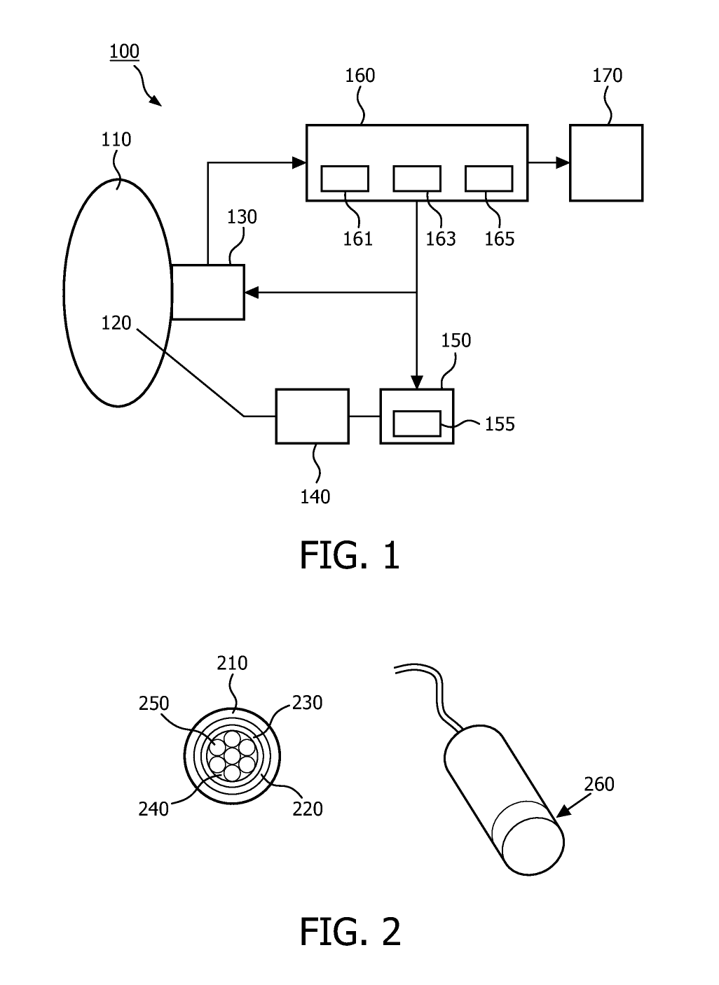

FIG. 1 shows a schematic diagram of a system 100 for navigating the insertion of a needle 120 moving in a region of a subject 110 in accordance with an embodiment of the present invention. The system 100 is configured to monitor the location of the needle 120, with the needle tip location being enhanced in the image displayed to a viewer, and at the same time monitor physiological indices of tissue into which the needle is to be inserted, during the time that the needle moves in the region. The subject 110 may be a human being, animals or inanimate objects. The needle 120 may be termed differently in accordance with other terminologies which refer to it as a line-segment shape instrument.

As shown in FIG. 1, the monitoring system 100 includes a needle 120, an US transducer 130, a coupler 140, an optical signal generating device 150, a processor 160 and a display 170.

The needle 120 is specially designed for the system 100. According to an embodiment of the present invention, the needle 120 includes a shell with needle shape, an optical dome disposed at a tip of the shell to form an inner needle space closed at the end of the tip; and an optical core built in the inner needle space, wherein the optical dome forms a lens for radiating optical signals transferred by the optical core out of the needle. As shown in FIG. 2, the needle 200, which is the needle 120 of FIG. 1, may be composed, viewed from outside to inside, of four layers, that is, shell 210, buffer 220, cladding 230 and optical core 240, respectively. The shell 210 may be made of metal. The optical core 240 may be composed of fiber 250. For example, inside the optical core, a bundle of multimode fibers with a higher coupling efficiency and a larger diameter of the fiber core (relative to the signal mode fiber) may be selected as the media to transfer optical signals such as laser pulses. An optical dome 260 may be coupled with the shell 210 or integrated in the shell 210 to form an inner needle space closed at the end of the tip of the needle. The optical dome 260 may be wrapped with a wide-band anti-refection film, for example, PMMA (polymethyl methacrylate) nanometer material made into monolayer or multilayer film, to address the optical signal transferred by the optical core out of the tip of needle into the tissue around or in front of the tip, that is to say, the optical signal radiates from the tip of the needle in the needle movement direction.

The optical signal generating device 150 may be a currently available laser system. For example, the device 150 may be an integrated, tunable laser system (like Phocus high energy (HE) near infrared ray (NIR) laser system using optical parametric oscillator (OPO) technology). In an example, the laser system may provide a 10-20 Hz repetition frequency and a 5-10 nanometer (nm) pulse duration.

The optical signal generating device 150 may include a wavelength tuning unit 155, through which the wavelength of the output optical signal can be tuned. In an example, the wavelength may be tuned in the range of 410 nm to 2100 nm. A series of spectrums specified by the aim of differentiating the elimination coefficients of the targeting tissues to be irradiated may be emitted by turns. In another example, the wavelength tuning unit may also be operated manually by the user to achieve a specified laser wavelength. In another example, the wavelength tuning unit may communicate the current wavelength information to the processor 160 to present the wavelength signature in the displayed image.

The coupler 140 may be used to connect the optical core of the needle with the output of the optical signal generating device 150. In another example, the optical core of the needle may be incorporated with the output of the laser system 150 without the coupler 140. The optical signal with a certain wavelength produced by the laser system 150 is transferred by the optical core of the needle 120 out of the dome into the area or tissue into which the needle is to be inserted, and an US signal is produced in the area in response to the radiating optical signal due to the PA effect. Hereinafter, for the sake of description, the US signal produced may be referred to as PA signal due to the PA effect.

The US transducer 130 may be an array of transducers for transmitting US signals into a region of the subject 110 and receiving corresponding reflected US signals in response to the transmitted US signals, and receiving the PA signal in response to the optical signal radiating into the tissue of the subject. The US transducer 130 may be used for both a conventional US imaging mode and the PA imaging mode. In the conventional US imaging mode, the transducer 130 may work as both transmitter and receiver. In the PA imaging mode, a synchronizing signal may be used to direct the transducer 130 to work as receiver only when the laser system is directed to transmit optical signals such as laser pulses from the needle tip into the tissue. The transducer 130 can convert the reflected US or PA signals to electrical signals, which present the radio frequency (RF) signal, and transmit the electrical signals to the processor 160.

The processor 160 may (e.g., with appropriate software and/or electronics) process the received RF signals to determine a resultant image (e.g., intensities of pixels for an image) and transmit the image to a display 170 for displaying to the viewer.

The processor 160 may include an image reconstruction module 161, a synchronization module 163 and a processing module 165.

According to an embodiment of the present invention, the synchronization module 163 controls the operation of the US transducer 130 and the optical signal providing device 150 in measurement cycles. A measurement cycle includes an US measurement sub-cycle, in which the US transducer 130 transmits an US signal into a region of a subject 110 into which the needle 120 is being inserted and receives an US signal reflected in the region in response to the transmitted US signal, and at least one PA measurement sub-cycle, in each of which the optical signal generating device 150 transmits an optical signal having a different wavelength from the dome of the needle into an area of the subject 110 and the US transducer receives the PA signal induced in the area in response to the optical signal without transmitting.

The transducer 130 provides the received US or PA RF signal to the processor 160. According to an embodiment of the present invention, the image reconstruction module 161 may reconstruct an US image from the US RF signal and reconstruct at least one PA image from the at least one PA RF signal received in the at least one PA measurement sub-cycle. The reconstruction may be performed through a sum-and-delay beam-forming algorithm which is known in the art. In an example, the processing unit 165 may combine the US image and some of the PA images to display to the viewer. Since the transducer 130, where both the reflected US signal and the induced PA signals are received, is hold at the same place during the needle insertion, the US image and the PA image are automatically registered at the same time instant without additional algorithms. The visible image for the viewer may be a fused image in between the US image and the PA image.

The reconstructed PA images contain the information of the needle localization and pathologic properties of the tissue in the irradiated area, especially in the microenvironment in the needle movement direction due to the small angle of spread of the radiated optical beams that is also called the directional stability of the laser pump. Accordingly, only the tissue around or in front of the needle tip, along its path of movement, will be irradiated and induced to generate the PA signal, and the location of the needle tip is naturally mapped into the PA image. Moreover, pathologic indices such as oxyhemoglobin, deoxyhemoglobin, carbonized tissue, and some qualitative measurements for lesions may be determined by the processing unit 165 according to absorption of respective optical signals having unique wavelengths in the tissue being irradiated, and the absorption of an optical signal may be determined from the PA signal induced in the tissue in response to the optical signal or may be determined from the PA image reconstructed from the PA signal. The processing unit 165 may present the pathologic indices in the image to be displayed as the needle moves to the potentially different biological tissues in the subject. Since the optical signal from the needle tip irradiates the tissue to be punched next in the case of rigid needle insertion, the monitoring system is able to identify the pathologic indices of the tissue before the needle is inserted into it, and can thus foresee the tissue structure such as the blood structure along the insertion direction so as to avoid any damage to even the tiniest vessel. In this way, the monitoring system may also indicate whether the needle is really placed inside the tumor or not, and measure the margin of the tumor for the tumor size calculation finished in the navigation step.

It should be understood that the modules 161-165 as shown in FIG. 1 may be implemented in a processor, for example, the processor 160, or in several hardware components, for example, the image reconstruction module 161 may be implemented in a dedicated processing unit such as a Digital Signal Processor (DSP) or an Application Specific Integrated Circuit (ASIC) or the like designed specifically for US image reconstructions, and the synchronization module 163 may be implemented in a controller or a general purpose processor which controls the operation of the components of the system, and the processing module 165 may be implemented in a general purpose processor, controller or the like.

It should be understood that the modules 161-165 as shown in FIG. 1 may be implemented in software as a computer program product; the functions of the modules may be stored on, or transmitted as program instructions or a code, on a computer-readable medium. Computer-readable media include any medium that facilitates transfer of a computer program from one place to another and that can be accessed by a computer. By way of example, the computer-readable media may include RAM, ROM, EEPROM, CD-ROM or other optical disk storage, magnetic disk storage or other magnetic storage devices, or any other medium that can be used to carry or store a desired program code in the form of instructions or data structures and that can be accessed by a computer.

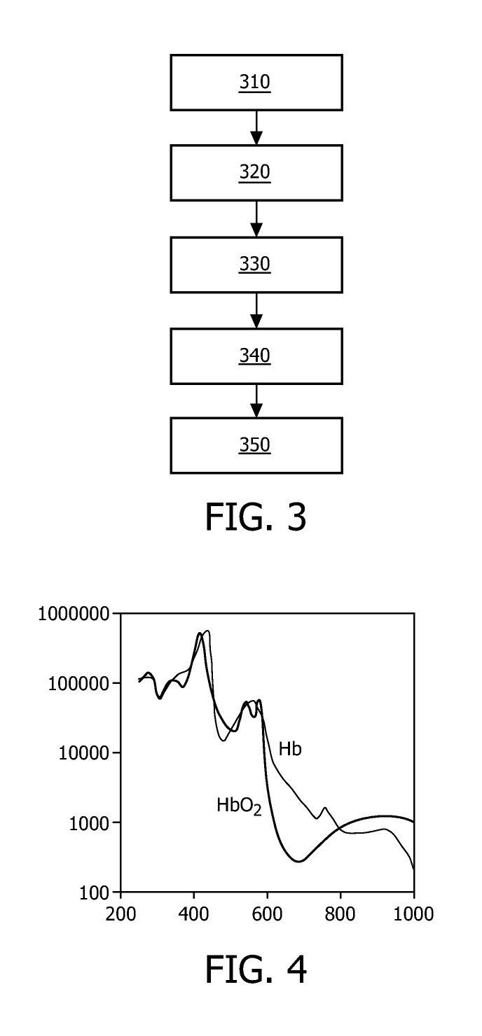

FIG. 3 is a flowchart of the method of navigating the insertion of a needle in a subject in accordance with an embodiment of the present invention. The method is implemented in the system 100, and particularly by the processor 160 or the modules 161-165 therein.

In the method, at least one of three monitoring modes may be activated, that is, needle tracking mode, damage prevention mode and qualitative tissue measurement mode. In tissue, the spatial distribution of the absorption coefficient is determined by the local concentration of the major tissue chromophores, such as oxyhemoglobin, deoxyhemoglobin, lipids and water. The absorption of each chromophore has a characteristic wavelength dependence, which allows spectroscopic information to be obtained by making PA measurements at different excitation wavelengths. According to an embodiment of the present invention, different wavelengths of optical signals may be selected for the three monitoring modes.

For the sake of description, it is assumed that all three modes are activated in the illustrated method. It should be understood that not all modes need to be activated to implement the present invention. And the steps illustrated in FIG. 3 are not necessarily performed in the shown order; some steps may be performed in parallel rather than sequentially. For example, the applying and receiving of signals in a current sub-cycle and the processing of the signals received in a previous sub-cycle may be performed in parallel.

In an US measurement sub-cycle of a measurement cycle, an US signal may be applied to a region of the subject 110 during the time that the needle 120 moves in the region and an US signal reflected in the region in response to the applied US signal may be received, and in each of at least one PA measurement sub-cycle of the measurement cycle, an optical signal having a unique wavelength may be applied from the dome of the needle into an area of the subject, and the PA signal induced in the area in response to the optical signal may be received (step 310).

In the US measurement sub-cycle of the measurement cycle, the synchronization module 163 or the processor 160 may control the US transducer 130 to transmit the US signal into the region of the subject in which the needle is moving and receive the reflected US signal (step 310).

After receiving the reflected US signal, the image reconstruction module 161 or the processor 160 may reconstruct a conventional US image from the received US RF signal (step 320).

The first PA measurement sub-cycle may be used for the needle tracking mode in which tracking the location of the needle takes place. In the first PA measurement sub-cycle of the measurement cycle, the synchronization module 163 or the processor 160 may control the optical signal generating device 150 to transmit a first optical signal having a first wavelength from the dome of the needle into the area of the subject, and control the US transducer 130 to receive a first PA signal induced in the area in response to the first optical signal (step 310).

After receiving the first PA signal, the synchronization module 163 or the processor 160 may reconstruct a PA image from the first PA RF signal (step 320). The PA image may present the location of the needle tip.

According to an embodiment of the present invention, a short wavelength•a in the range of 200-400 nm may be selected as the first wavelength for the use of the needle tracking mode since its optical penetration depth is extremely small, namely, the size of the irradiated area which absorbs the first optical signal is compressed into a spot. Then the needle tip can be visualized as a bright spot in the PA image. It should be understood that the range of 200-400 nm is a preferred option for the needle tip tracking, but wavelengths outside this range are also applicable for this purpose.

The processing module 165 or the processor 160 may fuse the PA image obtained in the needle tracking mode and the US image to achieve a fused image (step 350) to be displayed on the display 170.

The second PA measurement sub-cycle may be used for the damage prevention mode. In the second PA measurement sub-cycle of the measurement cycle, the synchronization module 163 or the processor 160 may control the optical signal generating device 150 to transmit a second optical signal having a second wavelength from the dome of the needle into the area of the subject, and control the US transducer 130 to receive a second PA signal induced in the area in response to the second optical signal (step 310).

After receiving the second PA signal, the image reconstruction module 161 or the processor 160 may reconstruct a second PA image from the second PA RF signal (step 320). It should be understood that in the damage prevention mode the reconstruction of the second PA image is not necessarily performed.

The processing module 165 or the processor 160 may determine the concentration of a chromophore, whose absorption property depends on the second wavelength, in the area according to the received PA signal (step 330). At step 330, the processing module 165 or the processor 160 may compare the concentration of the chromophore determined at the current needle tip location to at least one of those determined at previous needle tip locations, and trigger an alarm to a viewer when the comparison indicates a sudden change of the concentration of the chromophore determined at the current needle tip location. The sudden change of the concentration of the chromophore indicates a different tissue is about to be touched by the needle tip, and thus an alarm may be presented to prompt the viewer to take care.

The absorption of optical signals increases toward shorter wavelengths due to protein absorption, and toward longer wavelengths due to water absorption. According to an embodiment of the present invention, since, in the 400-600 nm range, absorption by hemoglobin is very strong and residual hemoglobin staining of vessel walls is a strong absorber of the optical signal with such a wavelength, a•b may be selected in this range as the second wavelength to detect the possibility of blood vessel existence.

From the second PA signal or the second PA image, which both contain the absorption information of the second optical signal in the irradiated area, the locally absorbed energy density in the area may be achieved. And the local concentration of the hemoglobin may be determined according to the absorbed energy density. That is to say, the concentration of the hemoglobin may be determined from the second PA signal or the second PA image, and generally the concentration of a chromophore in an area may be determined from the PA signal induced in the area in response to an optical signal having a wavelength related to the chromophore; this is known in the art. The derived local concentration of the hemoglobin, which is proportional to the absorbed energy density, varies with the positions of the needle tip. The processing module 165 or the processor 160 may compare the derived local concentration of the hemoglobin at the current needle tip location with the values thereof at previous needle tip positions. If the current concentration value suddenly exhibits an increase, the processing module 165 or the processor 160 may give an alarm that the needle tip is approaching the blood vessel.

The processing module 165 or the processor 160 may fuse the concentrations of the chromophore such as the hemoglobin determined at different needle tip locations in the US image (step 350) to be displayed.

The third and fourth PA measurement sub-cycles may be used for the qualitative measurement mode. In the third and fourth PA measurement sub-cycles of the measurement cycle, the synchronization module 163 or the processor 160 may control the optical signal generating device 150 to transmit consecutively a third optical signal having a third wavelength and a fourth optical signal having a fourth wavelength from the dome of the needle into the area of the subject, and direct the US transducer to consecutively receive a third and a fourth PA signal induced in the area in response to the third and the fourth optical signal (step 310).

After receiving the third and the fourth PA signal, the image reconstruction module 161 or the processor 160 may reconstruct a third and a fourth PA image from the third and the fourth PA RF signal respectively (step 320). It should be understood that in the qualitative measurement mode the reconstruction of the third and the fourth PA image is not necessarily performed.

The processing module 165 or the processor 160 may determine concentrations of endogenous chromophores such as oxyhemoglobin (HbO2) and deoxyhemoglobin (Hb) in the area according to the received third and fourth PA signals, and determine pathological information such as oxygen saturation (SO2) of blood according to the concentrations of the chomophores (step 340). As is known, the SO2 may be calculated as SO2=C_{HbO2}/(C_{HbO2}+C_{Hb}), where C_{HbO2} and C_{Hb} are concentrations of HbO2 and Hb.

As shown in FIG. 4, HbO2 and Hb exhibit different absorption spectra that are normally represented in terms of molar extinction coefficients. In FIG. 4, the horizontal axis refers to wavelength (nm), the vertical axis refers to Molar extinction coefficient (cm⁻¹M⁻¹). According to an embodiment of the present invention, the third and the fourth wavelength may be selected to be 940 nm and 660 nm because of the big difference in extinction coefficient between Hb and HbO2 at these two spectra, which is used to obtain the concentrations of HbO2 and Hb in the area irradiated by the third and the fourth optical signal. It should be understood that the third and the fourth wavelength are not limited to 940 nm and 660 nm. Other wavelengths may be selected as long as the molar extinction coefficients of Hb and HbO2 at the third and the fourth wavelength enable the concentrations of HbO2 and Hb to be precisely derived by the optical absorption values measured from PA signals. In practice, wavelengths may be selected to derive the concentrations of HbO2 or Hb as long as the difference in molar extinction coefficients of Hb and HbO2 at each of the wavelengths is measurable.

It should be understood that the pathologic information is not limited to the SO2 of blood. Other biochemical parameters to be considered as pathologic information for tissue can be measured in the same way in terms of other spectra applied.

The processing module 165 or the processor 160 may fuse the pathological information, such as SO2 of blood, determined at the current needle tip location or different needle tip locations, on the US image (step 350) to be displayed.

As stated above, in a measurement cycle, the processing module 165 or the processor 160 may fuse the location of the needle tip, the concentration of the hemoglobin in the area to be punctured and the SO2 of blood in the area on the US image to achieve a fused image, and send the fused image to the display 170 for displaying it to the viewer. Therefore, the display 170 displays the dual-modality fused image in a measurement cycle by measurement cycle manner, which carries both the needle tip position and the histo-pathological information analyzed from optical absorption indices in each functional mode.

It should be noted that the above-mentioned embodiments illustrate rather than limit the invention and that those skilled in the art will be able to design alternative embodiments without departing from the scope of the appended claims. In the claims, any reference signs placed between parentheses shall not be construed as limiting the claim. The word “comprising” does not exclude the presence of elements or steps not listed in a claim or in the description. The word “a” or “an” preceding an element does not exclude the presence of a plurality of such elements. In the system claims enumerating several units, several of these units can be embodied by one and the same item of software and/or hardware. The usage of the words first, second and third, et cetera, does not indicate any ordering. These words are to be interpreted as names.

1. (canceled)

2. (canceled)

3. A needle navigation system comprising
a needle comprising
a shell with a needle shape,
an optical dome disposed at a tip of the shell to form an inner needle space closed at the end of the tip,
an optical core built in the inner needle space,
and the optical dome forms a lens for radiating optical signals transferred by the optical core out of the needle;
an optical signal generating device, wherein at least one optical signal output of the optical signal generating device is coupled to the optical core;
an ultrasound (US) transducer; and
a processor adapted to direct the US transducer to transmit a US signal into a region of a subject in which the needle is moving and receive a US signal reflected in the region in response to the transmitted US signal in a US measurement sub-cycle of a measurement cycle, and reconstruct a US image from the US signal received in the US measurement sub-cycle, and direct the optical signal generating device to transmit a first optical signal having a first wavelength from the dome of the needle into the area of the subject in one of the at least one photo-acoustic (PA) measurement sub-cycle, direct the US transducer to receive a PA signal induced in the area in response to the first optical signal in the PA measurement sub-cycle, reconstruct a PA image from the received PA signal, and fuse the PA image and the US image to achieve a fused image to be displayed, wherein the first wavelength is selected in a range of 200-400 namometer (nm), wherein the processor is further adapted to direct the optical signal generating device to transmit a second optical signal having a second wavelength from the dome of the needle into the area of the subject in one of the at least one PA measurement sub-cycle, direct the US transducer to receive a PA signal induced in the area in response to the second optical signal in the PA measurement sub-cycle, determine the concentration of a chromophore, whose absorption property depends on the second wavelength, in the area according to the received PA signal, compare the concentration of the chromophore determined at the current needle tip location to at least one of those determined at previous needle tip locations, and trigger an alarm to a viewer when the comparison indicates a sudden change of the concentration of the chromophore determined at the current needle tip location, wherein the second wavelength is selected in a range of 400-600 nm.

4. (canceled)

5. (canceled)

6. (canceled)

7. The needle navigation system according to claim 1, wherein the chromophore is hemoglobin, and the processor is further adapted to trigger an alarm when the comparison indicates a sudden increase of the concentration of the chromophore determined at the current needle tip location, and present the concentrations of the chromophore determined at different needle tip locations on the US image.

8. The needle system according to claim 1, wherein the processor is further adapted to direct the optical signal generating device to transmit consecutively a third optical signal having a third wavelength and a fourth optical signal having a fourth wavelength from the dome of the needle into the area of the subject in two of the at least one PA measurement sub-cycle, direct the US transducer to consecutively receive PA signals induced in the area in response to the third and fourth optical signals in the two PA measurement sub-cycles, determine the concentration of oxyhemoglobin (HbO2) and the concentration of deoxyhemoglobin (Hb) in the area according to the received PA signals, determine oxygen saturation (SO2) of blood in the area according to the concentration of HbO2 and the concentration of Hb, and present the SO2 on the US image to be displayed.

9. The needle navigation system according to claim 8, wherein the molar extinction coefficients of Hb and HbO2 at the third and fourth wavelengths enable the concentrations of Hb and HbO2 to be precisely derived by the optical absorption values measured from PA signals.

10. The needle navigation system according to claim 9, wherein the third and fourth wavelengths are selected to be 940 nm and 660 nm, respectively.

11. A needle navigation method when a needle, comprising a shell with a needle shape, an optical dome disposed at a tip of the shell to form an inner needle space closed at the end of the tip, an optical core built in the inner needle space, and the optical dome forms a lens for radiating optical signals transferred by the optical core out of the needle moves in a region of a subject, the method comprising:
applying an ultrasound (US) signal to the region in an US measurement sub-cycle of a measurement cycle, and receiving an US signal reflected in the region in response to the applied US signal;
reconstructing an US image from the US signal received in the US measurement sub-cycle;
applying a first optical signal having a first wavelength from the dome of the needle into the area of the subject in one of the at least one PA measurement sub-cycle;
receiving a PA signal induced in the area in response to the first optical signal in the PA measurement sub-cycle;
reconstructing a PA image from the received PA signal; and
fusing the PA image and the US image to achieve a fused image to be displayed,
applying a second optical signal having a second wavelength from the dome of the needle into the area of the subject in one of the at least one PA measurement sub-cycle;
receiving a PA signal induced in the area in response to the second optical signal in the PA measurement sub-cycle;
determining the concentration of a chromophore, whose absorption property depends on the second wavelength, in the area according to the received PA signal;
comparing the concentration of the chromophore determined at the current needle tip location to at least one of those determined at previous needle tip locations; and
triggering an alarm to alert a viewer when the comparison indicates a sudden change of the concentration of the chromophore determined at the current needle tip location.

12. (canceled)

13. (canceled)

14. The needle navigation method according to claim 11, further comprising:
presenting the concentrations of the chromophore determined at different needle tip locations on the US image.

15. The needle navigation method according to claim 11, further comprising:
applying consecutively a third optical signal having a third wavelength and a fourth optical signal having a fourth wavelength from the dome of the needle into the area of the subject in two of the at least one PA measurement sub-cycle;
receiving PA signals induced in the area in response to the third and fourth optical signals in the two PA measurement sub-cycles;
determining the concentration of oxyhemoglobin (HbO2) and the concentration of deoxyhemoglobin (Hb) in the area according to the received PA signals;
determining the oxygen saturation (SO2) of blood in the area according to the concentration of HbO2 and the concentration of Hb; and
presenting the SO2 on the US image to be displayed.
